(19) United States
(12) Patent Application Publication
(10) Pub. No.: US 2014/0030308 A1
(43) Pub. Date: Jan. 30, 2014

(54) IMPLANTS AND PROCEDURES FOR PROMOTING AUTOLOGOUS STEM CELL GROWTH

(71) Applicant: Steven S. Crohn, Paradise Valley, AZ (US)

(72) Inventor: Steven S. Crohn, Paradise Valley, AZ (US)

(21) Appl. No.: 13/938,199

(22) Filed: Jul. 9, 2013

Related U.S. Application Data
(60) Provisional application No. 61/676,106, filed on Jul. 26, 2012.
(60) Provisional application No. 61/691,067, filed on Aug. 20, 2012.

(51) Int. Cl. A61F 2/82; A61K 38/19; A61K 31/395; A61K 38/18

(52) U.S. Cl. CPC A61F 2/82; A61K 38/1866; A61K 38/193; A61K 38/195; A61K 31/395
USPC 424/423; 514/8.1; 424/85.1; 514/7.6; 623/1.42

(57) ABSTRACT

A biologically engineered stent for treating patients suffering from acute myocardial infarction/ischemia. The stent is inserted in a vessel upstream to and proximal the damaged muscle/ischemic area. The stent elutes Stromal Derived Factor (SDF1)/CXCR4 complex and/or Vascular Endothelial Growth Factor (VEGF) to attract autologous stem cell for the repair of damaged myocardium or tissues and inducing vascularization (creation of collateral vessels) to the ischemic area. The SDF1/CXCR4 acts as a homing mechanism for stem cells. Stem cell mobilizing agents such as Gm-CSF, GCSF and Plerixafor, as a CXCR4 blocker, may be added systemically to assist in stem mobilization. A protocol consisting of multiple doses of Gm-CSF or GCSF may be given in order to mobilize stem cells from the patient. Optionally, stem cells may be injected into the patient. The treatment stimulates repair and improves survival of damaged myocardium and prevents ventricular remodeling.

### RELATED APPLICATIONS

This application claims the benefit under 35 USC Section 119(e) of U.S. Provisional Application No. 61/676,106 filed on Jul. 26, 2012 and U.S. Provisional Application No. 61/691,067 filed on Aug. 20, 2012, the entire disclosures of which are incorporated herein by reference.

### BACKGROUND OF THE INVENTION

1. Field of the Invention

Broadly, this invention relates to a method of treating damaged cardiac muscle or myocardium, especially cardiac muscle damaged by myocardial infarction, to stimulate survival and repair of damaged myocardium and prevent myocardial remodeling.

This invention also relates to implants, and in particular stents, which are medical devices used to open and maintain patency in vessels of the body, for example to maintain blood flow through diseased blood vessels.

More specifically, the invention relates to biologically engineered stents (BES) that are useful for localized delivery of therapeutic drugs, molecules and cells to the walls of damaged vessels and muscles, following implantation of the stent in the vessel proximal the damage. Such biologically engineered stents (BES) can deliver drugs to cells in the walls of stented vessels, thereby promoting local production of therapeutic factors that attract and enhance the formation of endothelium in the stented vessel.

Still more specifically, this invention relates to an implant, for example a polymer implant, most preferably a stent, that is suitable for implantation in the myocardial circulatory system and capable of delivering components that act as homing mechanisms for stem cells and/or signaling factors that signal the recruitment of vascular progenitor cells.

Most preferably the implant is a stent impregnated with Stromal Derived Factor (SDF1)/CXCR4 complex and Vascular Endothelial Growth Factor (VEGF) to promote autologous stem cell growth around the stent and revascularization. The implant may be coated, impregnated, infused or otherwise coupled with such components. Optionally, the stent may be coated with additional types of drugs or therapeutic materials such as antibiotics, thrombolytics, anti-thrombotics, anti-inflammatories, cytotoxic agents, anti-proliferative agents, vasodilators, gene therapy agents, radioactive agents, immunosuppressants, chemotherapeutics, other endothelial cell attractors or promoters and/or stem cells.

Such materials may be coated over all or a portion of the surface of the stent or the stent may have a porous structure or include apertures, holes, channels, or other features in which such materials may be deposited. Embodiments of BES include xenografts, allografts or isografts comprising sleeve-like natural matrices derived from vessels of animal and human subjects including postmortem human donors and medically produced grafts or implants as well as polymeric stents.

A protocol consisting of multiple doses of Gm-CSF or GCSF may be given after stent placement in order to mobilize stem cells maximally from the bone marrow of the patient. The mobilization from bone marrow of endothelial progenitor cells—autologous stem cells from the patient, using either Gm-CSF or GCSF, substantially increases the number of stem cells homing in to such stents. Optionally, stem cells may also be injected into the patient.

2. Description of the Related Art

Myocardial infarction, i.e., the formation of an infarct or an area of dead heart muscle, occurs when the blood supply to the heart is interrupted, which can be the result of occlusion (blockage) of a coronary artery. Acute myocardial infarction (AMI) occurs as the result of sudden blockage of blood supply to the heart. Irreversible death of heart muscle begins to occur if the blood supply is not re-established quickly enough (e.g., within 20 to 40 minutes).

If impaired blood flow to the heart lasts long enough, heart cells die, via necrotic and/or apoptotic cellular pathways, do not grow back and a collagen scar forms in their place. This can result in permanent damage to the heart, and scar tissue also puts the patient at risk for potentially life threatening arrhythmias, and/or may result in the remodeling of the myocardium and formation of a ventricular aneurism. Left Ventricular Wall abnormalities may result as well.

Diseases of the heart, such as MI, are the leading cause of death for both men and women. It is estimated that coronary heart disease is responsible for 1 in 5 deaths in the U.S. About 1.2 million people in the U.S. suffer a new or recurrent coronary attack every year and of them, approximately 400,000 die as a result of the attack.

Acute myocardial infarction is very common in the United States and globally. It is known that following a myocardial infarction, the acute loss of myocardial muscle cells result in a cascade of events causing an immediate diminution of cardiac function, with the potential for long term persistence or death. The extent of myocardial cell loss is dependent on the duration of coronary artery occlusion, existing collateral coronary circulation and the condition of the cardiac microvasculature. Because myocardial cells have only a minimal ability to regenerate, myocardial infarction usually leads to permanent cardiac dysfunction due to contractile-muscle cell loss and replacement with nonfunctioning fibrotic scarring. Moreover, compensatory hypertrophy of viable myocardium leads to microvascular insufficiency that results in a further demise in cardiac function.

Among survivors of myocardial infarction, residual cardiac function is influenced most by the extent of ventricular remodeling, i.e., changes in size, shape, and function, typically a decline in function, of the heart after injury. Alterations in ventricular topography, i.e., meaning the shape, configuration, or morphology of a ventricle, occur in both infarcted and healthy cardiac tissue after myocardial infarction. Ventricular dilatation, i.e., a stretching, enlarging or spreading out of the ventricle, causes a decrease in global cardiac function and is affected most by the infarct size, infarct healing and ventricular wall stresses.

Recent efforts to minimize remodeling have been successful by limiting infarct size through rapid reperfusion, i.e., restoration of blood flow, using thrombolytic agents and mechanical interventions, including, but not limited to, placement of a stent, along with reducing ventricular wall stresses by judicious use of pre-load therapies and proper after-load management.

One example of a stent is a small, coiled wire-mesh tube that can be inserted into a blood vessel, such as an artery in the heart, which may be used to open a narrowed or clotted blood vessel. Mesh-like stents allow blood vessel cells to grow through the mesh lining the stent and helping to secure it.

Stents are commonly used during angioplasty and other revascularization procedures. Balloon angioplasty is often used to insert stents, although sometimes stents are placed without the use of a balloon. The stent may be expanded using a small balloon during the angioplasty procedure. When the balloon inside the stent is inflated, the stent expands and presses against the walls of the artery, which traps any fat and calcium buildup against the walls of the artery, allows blood to flow through the artery, and helps prevent the artery from closing again (restenosis). The stent may also help prevent small pieces of plaque from breaking off and causing downstream occlusion and myocardial infarction. The balloon is then deflated and removed, leaving the stent in place.

The stent resists the re-stenosis caused by vascular delamination or elastic recoil, thus significantly decreases the complications of acute or subacute ischemia. However, long-term implantation of stents stimulates the immigration and proliferation of smooth muscle cells, resulting in intimal hyperplasia which in turn leads to re-stenosis.

Though existing drug-coated stents, as compared to uncoated stents, have greatly improved the treatment of vascular re-stenosis, results from long-term analysis show that drug-coated stents do not increase the survival rate of patients and might result in some adverse effects, such as delayed thrombus that may be fatal, and chronic inflammatory reactions resulted from bio-inert polymers.

Stents are also provided that comprise a drug or agent that is released from the stent at a controlled rate and concentration over a specified time interval upon insertion, e.g. at a site of an acute coronary artery occlusion upstream of the site of acute myocardial infarction or ischemia. These drug-eluting stents are stents that are coated with agents. These agents may be cardioprotective agents or other agents. Drug-eluting stents are well known in the art. The cardioprotective agents and other agents, alone or in combination, can be combined with organic or inorganic carrier molecules, elution factors, solvents, salts, biopolymers, synthetic polymers and applied to the stent to generate a coated stent. Stent coating is known in the art and may involve immersion of the stent in a solution or may involve spray coating.

Drug-eluting stents may be coated with cells, such as endothelial cells engineered to express cellular factors that have, for example, cardioprotective, angiogenic, anti-thrombotic, antiplatelet, anticoagulant, antimicrobial, anti-inflammatory, antimetabolic, and/or vasoreactive effects, or may be directly coated with genes encoding polypeptides exerting similar effects. Drug-eluting stents may be coated with agents such as cardioprotective agents, angiogenic agents, anti-thrombotic agents, antiplatelet agents, anticoagulant agents, antimicrobial agents, anti-inflammatory agents, antimetabolic agents, and/or vasoreactive agents.

Regardless of these interventions, a substantial percentage of patients experience clinically relevant and long-term cardiac dysfunction after myocardial infarction. Despite revascularization of the infarct related arterial circulation and appropriate medical management to minimize ventricular wall stresses, a significant percentage of patients experience ventricular remodeling, permanent cardiac dysfunction, and consequently remain at an increased lifetime risk of experiencing adverse cardiac events, including death.

Although catheter-based revascularization or surgery-based treatment approaches have been successful in restoring blood flow to ischemic myocardium in the majority of cases, the treatments are inadequate for a significant number of patients who remain incompletely revascularized. The ramifications of treatment limitations may be significant in patients who have large areas of ischemic, but viable myocardium jeopardized by the impaired perfusion supplied by vessels that are poor targets for conventional revascularization techniques. Treatment alternatives, including mechanical approaches such as percutaneous transluminal myocardial revascularization, and the like, have not produced encouraging results. Gene therapy using adenoviral vectors to augment cytokine production and, therefore, promote angiogenesis has shown promise, but this therapy has limitations and has not yet emerged as the optimal treatment for these patients. Therefore, therapeutic angiogenesis has attracted many researchers attempting to discover a way to circumvent the burden of chronic myocardial ischemia.

It is known that the production of blood vessels is accomplished by two main processes: angiogenesis and vasculogenesis. Angiogenesis refers to the production of vascular tissue from fully differentiated endothelial cells derived from pre-existing native blood vessels. Angiogenesis is induced by complex signaling mechanisms of cytokines including vascular endothelial growth factor (VEGF) and other mediators. “Therapeutic angiogenesis” refers to utilizing cytokines derived from recombinant therapy, using proteins, cells or angiogenic factors or any combination thereof to induce or augment collateral blood vessel production in patients with ischemic vascular diseases. “Therapeutic vasculogenesis” refers to neogenesis of vascular tissues by introduction of exogenous endothelial producing cells into the patient.

It is also known that the delivery of stem cells to an affected organ or tissue can regenerate tissue or organ function. However, stem cells are not well retained in the organ targeted for tissue regeneration even when the stem cells are directly injected into the tissue of the injured organ. Imaging studies in humans and animals have demonstrated that most of the delivered stem cells can be found within the spleen within an hour after stem cell injection.

Patients treated with stem cells to elicit organ regeneration have demonstrated reductions in mortality and improvements in function following stem cell therapy, although the stem cell treatments do not generally restore the patient to their functional status prior to organ injury. Reductions in stem cell binding to the spleen and other lymphatics augment the numbers of circulating stem cells that can be attracted to the injured organ and thereby augment the degree of functional recovery induced by stem cell treatment of that patient.

The feasibility of using gene therapy to enhance angiogenesis has received recognition. VEGF-1 has been administered as a balloon gene delivery system. In addition, it has been reported that direct intramuscular injection of DNA encoding VEGF-1 into ischemic tissue induces angiogenesis, providing the ischemic tissue with increased blood vessels.

The use of endothelial progenitor cells (EPCs) as therapeutic agents for ischemia, including cardiac ischemia, has been investigated. There have been reports that EPCs isolated from peripheral blood can be augmented in response to certain cytokines and/or tissue ischemia. EPCs have been shown to home to, and incorporate into sites of neovascularization (i.e., sites of new blood vessel formation). Small molecule therapeutic agents have also been found to be useful in the treatment of ischemia. CXCR4 antagonists, originally developed for the treatment of HIV have recently been found to be effective in the treatment of ischemia, including ischemia associated with acute myocardial infarction

A number of angiogenesis techniques are known, including gene therapy and the use of growth factors such as vascular endothelial growth factor (VEGF) or basic fibroblast growth factor (bFGF) to induce or augment collateral blood vessel production to provide treatment for myocardial ischemia and/or peripheral vascular occlusive disease. These techniques rely on the availability of a resident population of mobilizable and hormone responsive vascular endothelial cells in the patient's circulation. However, there exists an age-related diminution of vascular endothelial cell number and function in adults. In particular, in older patients who are most likely to suffer from vascular problems, both central (i.e. coronary) and peripheral, the number of hormone responsive endothelial cells is reduced and the number of dysfunctional endothelial cells is increased. Moreover, administration of cytokines to mobilize sufficient patient-derived responsive cells may worsen cardiovascular pathophysiology secondary to leukocytosis and/or activation of pro-coagulant processes.

Therefore, an alternative therapy, that of supplying an exogenous source of endothelial progenitor cells (EPCs), may be optimal for cellular therapeutics to enhance vasculogenesis and collateralization around and downstream of the blocked or narrowed vessels to relieve ischemia. Clinical use of autologous patient-derived sources of stem cells is advantageous to avoid potential adverse allogeneic immune reactivity; however, the disadvantages include the need to subject the patient to stem cell collection at a time of active vascular disease.

Therefore, there is still a need to develop treatment modalities for both myocardial ischemia and peripheral vascular disease that can promote vasculogenesis in the ischemic tissue.

The following references are known by Applicant and may be related to the invention described and claimed herein:

US 2006/0136050 to Chu et al. describes means for ameliorating stent graft migration and endoleak using treatment site-specific cell growth promoting compositions in combination with stent grafts and coating of autologous growth factor compositions onto stent grafts prior to stent graft implantation. US 2006/0024347 to Zamora et al. describes coating a medical device, including a stent with one of many growth factors including VEGF and SDF-1.

US 2006/0233850 to Michal describes bioscaffolding stents with SDF-1 and VEGF.

US 2008/0233082 to Hoh et al. describes using a polymer composition with SDF-1 and VEGF for coating existing devices, e.g. carotid stents. The device can be used for vascular repair and includes a polymer incorporating signaling factors that signal the recruitment of vascular progenitor cells. The devices according to the invention can comprise a variety of devices, including stents (e.g., coronary stents, peripheral stents, carotid stents, intracranial stents, and aortic stent grafts) and aneurysm coils. For example, the polymer could be used to at least partially coat a pre-made stent or aneurysm coil.

US 2009/0228088 to Lowe et al. describes specifically structured prosthetic devices, e.g., stents, with a specific structure and geometry having improved flexibility, for use in the coronary and peripheral arteries as well as in other vessels and body lumens.

US 2009/0221683 to Losordo describes treating symptoms associated with tissue ischemia by administering a CXCR4 antagonist in combination with at least one nucleic acid encoding a morphogen or effective fragment thereof The CXCR4 inhibitor is formulated for systemic, preferably subcutaneous administration, and the nucleic acid coding a morphogen or an active fragment of a morphogen is formulated for injection into the ischemic tissue, preferably intramuscular injection, more preferably for pericardial or intracardiac injection. The invention can be used alone or in combination with other agents such as those that promote the proliferation of endothelial progenitor (EPC) cells. US 2009/0274687 to Young et al. describes a polymeric matrix in the form of a micro-particle, such as a micro-sphere wherein an anti-SDF-1 agent or CXCR4 antagonist is dispersed throughout the polymeric matrix and a microcapsule comprising an anti-SDF-1 agent or CXCR4 antagonist. A form of the polymeric matrix for containing an anti-SDF-1 agent or CXCR4 antagonist includes a stent.

US 2010/0161032 to Avellanet discloses a biologically engineered stent, e.g., double-walled, for blood vessels that can deliver drugs, for example, in the form of gene therapy vectors to cells in the walls of the stented vessel causing the cells to produce therapeutic factors that promote the formation of endothelium in the vessel. The delivery of two or more drugs is described. The reference describes a coating of the stent with at least one of SDF-1 or VEGF.

US 2010/0256153 to Frederich discloses use of VEGF receptors, VEGFA inhibitors, Plerixafor and SDF-1 as co-administered with DPP-IV inhibitor for treatment for coronary disease.

US 2010/0324276 to Sundaram et al. describes coating a stent with a polysaccharide composition that can include VEGF or CXCR4 antagonists.

US 2011/0014121 to Chen et al. describes endothelial progenitor cells (EPCs) mobilized following administration of the compounds or compositions that are modulators of CXCR. Optionally, EPC mobilization is induced by administration of compounds or compositions that are modulators of CXCR in conjunction with one or more of vascular endothelial growth factor (VEGF) or a VEGF agonist (including but not limited to a VEGF agonist antibody).

US 2011/0202125 to Luo describes artificial stents and production of such stents. The reference describes a microporous coated stent submerged in a solution of VEGF protein.

US 2011/0206688 to Bartelmez et al. describes the use of CXCR4 antagonist during SDF-1 treatment.

US 2011/0245915 to Caplice discloses a drug eluting stent for coronary circulation treatment having a coating of IGF-1 that is adapted to release IGF-1 over a period of up to 14 days. The stent may have a cardioprotective agent, e.g., cytokines, anti-coagulating agents, vessel spasticity minimizing agents, a vasodilator and an anti-inflammatory factor such as VEGF or SDF-1.

US 2011/0274744 to Picart et al. describes processes for coating a surface with a cross-linked polyelectrolyte multilayer film incorporating a protein, preferably a growth factor type protein. Growth factors incorporated into films includes SDF-1 and VEGF. The article coated can be blood vessel stents, tubing, angioplasty balloons, vascular graft tubing, prosthetic blood vessels, vascular shunts, heart valves, artificial heart components, pacemakers, pacemaker electrodes, pacemaker leads, ventricular assist devices, and numerous other medical devices.

US 2012/0035150 to Gaweco et al. discloses use of administering Macrophage Migration Inhibitory Factor, MIF inhibitors, inter alia, through a drug-eluting stent in combination with, inter alia, CXCR2 antagonists, VEGF-A, Plerixafor and/or numerous other therapeutic agents.

US 2012/0045435 to Deisher describes compositions and methods of inhibiting stem cell binding to organs and tissues, including the blocking of stem cell binding to germinal centers present in lymph tissue.

U.S. Pat. No. 6,676,937 to Isner et al. reports using gene therapy to enhance angiogenesis with VEGF-1 administered as a balloon gene delivery system. Transfer and expression of the VEGF-1 gene in the vessel wall subsequently augmented neovascularization in the ischemic limb, and the direct intramuscular injection of DNA encoding VEGF-1 into ischemic tissue induced angiogenesis, providing the ischemic tissue with increased blood vessels. The reference further describes coating with angiogenic proteins (VEGF) and vascularization modulating agent (SDF-1).

U.S. Pat. No. 7,470,538 to Laughlin et al. describes cell-based methods for the treatment of ischemia. Therapies are described for increasing blood flow to an ischemic tissue by promoting the formation of blood vessels. Cells are introduced, by infusion, into the patient that can differentiate into endothelial cells or that promote the differentiation of cells from the subject into endothelial cells. Administration of the cells to the subject is performed using an intra-arterial catheter, such as but not limited to a balloon catheter, or by using a stent. Such cells are stem cells and progenitor cells. The cells may be isolated from bone marrow, peripheral blood, umbilical cord cells or from other sources. Stents are used to deliver VEGF, SDF for ischemia treatment.

U.S. Pat. No. 7,775,469 to Poznansky et al. describe coating a medical material surface, e.g. a stent, with a fugetactic agent such as SDF-1 alpha or a CXCR-4 ligand. The medical material promotes migration of cells away from a transplanted tissue in a subject.

U.S. Pat. No. 8,088,370 and U.S. Pat. No. 7,794,705 to Pecora et al. discloses that chemokine stromal cell derived factor-1 (SDF-1), which is the ligand for the CXCR-4 chemokine receptor expressed by endothelial progenitor cells, plays a role in homing of cells to areas of ischemic damage. The reference further describes vascular endothelial growth factor (VEGF) inducing fibroblast proliferation, influencing extracellular matrix metabolism, and inducing angiogenesis. A pharmaceutical composition for the repair of vascular injury is described comprising a chemotactic hematopoietic stem cell product comprising an enriched population of CD34+ cells containing a subpopulation of cells having chemotactic activity. In some embodiments, this chemotactic activity is mediated by SDF-1, VEGF, and/or CXCR-4. The composition is administered with an infusion syringe, a ballon/delivery catheter, e.g., administering the composition via balloon catheterization into an infarcted artery. This reference requires that stents be placed prior to infusion of the chemotactic hematopoietic stem cell product.

U.S. Pat. No. 8,106,146 to Benz et al. describes a polyorthoester polymer that includes at least one therapeutic compound in the polymer backbone). The polymers may be formed into medical devices, such as vascular grafts, stents, pacemaker leads, heart valves, and the like, that are implanted in blood vessels or in the heart and devices for temporary intravascular use such as catheters, guide wires, and the like which are placed into the blood vessels or the heart for purposes of monitoring or repair. Biologically active agents, such as growth factors, receptors, and cytokines, may be included in the polymer, e.g., SDF-1 and VEGF.

Stewart et al. Pharmacokinetics and Pharmacodynamics of Plerixafor in Patients with Non-Hodgkin Lymphoma and Multiple Myeloma. Biology of Blood and Marrow Transplantation; Volume 15, Issue 1, Pages 39-46, January 2009.

Jean-Pierre Levesque et al. Disruption of the CXCR4/CXCL12 chemotactic interaction during hematopoietic stem cell mobilization induced by GCSF or cyclophosphamide. J. Clin Invest. 2003 Jan. 15; 111(2): 187-196.

A. F. Cashen et al. Mobilizing stem cells from normal donors: is it possible to improve upon G-CSF? Bone Marrow Transplantation (2007) 39, 577-588. doi:10.1038/sj.bmt.1705616; published online 19 Mar. 2007.

Carmen Urbich et al. Endothelial Progenitor Cells: Characterization and Role in Vascular Biology. Circ Res. 2004;95:343-353doi: 10.1161/01.RES.0000137877.89448.78.

Richardson J D et al., Optimization of the Cardiovascular Therapeutic Properties of Mesenchymal Stromal/Stem Cells-Taking the Next Step. Stem Cell Rev. 2012 Apr. 13.

Zhang G W et al. Transmyocardial drilling revascularization combined with heparinized bFGF-incorporating stent activates resident cardiac stem cells via SDF-1/CXCR4 axis. Exp Cell Res. 2012 Feb. 15;318(4):391-9. Epub 2011 Nov. 28.

Bone-marrow derived circulating vascular progenitor cells have been shown to migrate to sites of vascular injury. These circulating vascular progenitor cells, rather than local neighboring cells, seem to be the agents for vascular repair. Thus, it would be useful to have mechanism for promoting the recruitment of circulating vascular progenitor cells and thus improving the treatment of vascular conditions, such as those described above.

In general, the prior art has looked at increasing mobilizing stem cells, embedding stents with various molecules but has not managed to accomplish both by embedding stents with homing components for stem cells and injecting into the coronary artery concentrated stem cells. The entire disclosure of all of the aforedescribed references are incorporated herein by reference.

### SUMMARY OF THE INVENTION

It is a broad object of this invention to provide a biologically engineered implant or stent for promoting formation of vascular endothelium in a blood vessel into which it is inserted.

A more specific object of this invention to provide implants, and in particular stents that attract circulating EPCs and/or promote the formation of an endothelial layer over the area of stent implantation.

It is a further object, that upon implantation into a blood vessel, the stents of this invention promote the growth of an intact layer of endothelium over the stent and the adjacent vessel walls.

A further object of this invention to provide an implant, polymer implant, graft and transplanted tissue, and most preferably a stent, that is suitable for implantation in the myocardial circulatory system and capable of delivering components that act as a homing mechanism for stem cells and/or signaling factors that signal the recruitment of endothelial progenitor cells as well as cardiac progenitor cells.

These and other objects are achieved by an implant for implanting in a damaged vessel of patient. The implant comprises an amount of a gene therapy vector bonded to the implant for delivery of an effective treatment amount of the gene therapy vector to the damaged vessel.

More specifically, this invention is directed to a biologically engineered stent for implanting in a vessel proximal a damaged myocardium of a patient upstream thereof, to stimulate survival and repair of the damaged myocardium. The stent comprises an amount of a component selected from the group consisting of:
a. Stromal Derived Factor (SDF1)/CXCR4 complex, or
b. Vascular Endothelial Growth Factor (VEGF), or
c. Mixtures of a. and b.

The invention is further directed to the use of the implants and stents, and treatments of the patient subsequent to and/or prior to implantation with Gm-CSF or GCSF, stem cells and/or AMD3100 (1,1′-[1,4-phenylenebis(methylene)]bis-1,4,8,11-tetraazacyclotetradecane) or mimetics thereof.

Still more specifically, the stent is inserted in a vessel upstream to and proximal the damaged muscle/ischemic area. The stent elutes Stromal Derived Factor (SDFI)/CXCR4 complex and/or Vascular Endothelial Growth Factor (VEGF) to attract autologous stem cell for the repair of damaged myocardium or tissues and inducing vascularization (creation of collateral vessels) to the ischemic area. The SDF1/CXCR4 acts as a homing mechanism for stem cells. Stem cell mobilizing agents such as Gm-CSF, GCSF and Plerixafor, as a CXCR4 blocker, may be added systemically to assist in stem mobilization. A protocol consisting of multiple doses of Gm-CSF or GCSF may be given in order to mobilize stem cells from the patient. Optionally, stem cells may be injected into the patient. The treatment stimulates repair and improves survival of damaged myocardium and prevents ventricular remodeling.

### DESCRIPTION OF THE INVENTION

This invention is directed to methods and medical devices for the treating of damaged myocardium, especially myocardium damaged by myocardial infarction, to stimulate survival and repair of damaged myocardium and prevent ventricular remodeling.

This invention contemplates implants, and in particular stents, which are medical devices used to open and maintain patency in vessels of the body, for example to maintain blood flow through diseased blood vessels.

It has been established that the human body has a natural repair process for replacing lost or damaged endothelial cells in blood vessels. Cells known as “endothelial progenitor cells” (EPCs) are bone marrow-derived stem cells that circulate in the bloodstream and have the ability to home to blood vessel walls and differentiate into mature, functional endothelial cells that integrate into the endothelium.

Based on the above observations and principles, the implants, i.e., stents of this invention are designed to attract circulating EPCs, cardiac progenitor cells and/or to promote the formation of an endothelial layer over the area of stent implantation. More specifically, upon implantation into a blood vessel, the stents of this invention are designed to promote the growth of an intact layer of endothelium over the stent and the adjacent vessel wall underlying the struts of the stent.

Accordingly, one important aspect of the invention is a biologically engineered stent for promoting formation of vascular endothelium in a blood vessel.

More specifically, the invention contemplates biologically engineered stents (BES) that are useful for localized delivery of therapeutic drugs, molecules and cells to the walls of damaged vessels, following implantation of the stent in the vessel for promoting the formation of vascular endothelium in a blood vessel. Such biologically engineered stents (BES) can deliver drugs to cells in the walls of stented vessels, thereby promoting local production of therapeutic factors that attract and enhance the formation of endothelium in the stented vessel.

More specifically, this invention contemplates broadly any type implant, polymer implants, as well as grafts and transplanted tissues, and most preferably a stent, that is suitable for implantation in the myocardial circulatory system and capable of delivering components that act as a homing mechanism for stem cells and/or signaling factors that signal the recruitment of vascular progenitor cells.

Most preferably the implant is a stent impregnated with Stromal Derived Factor (SDF1)/CXCR4 complex and Vascular Endothelial Growth Factor (VEGF) to promote autologous stem cell growth around the stent as well as revascularization around and downstream of the stented area as well as attract and/or enhance a number of cardiac progenitor cells to the area of damaged myocardium.

Still more specifically, this invention is directly coating stents wherein the coating comprises bioactive proteins, in particular vascular stents impregnated with SDF-1 (stromal derived factor-1)/CXCR4 complex and VEGF (vascular endothelial growth factor) in order to use the homing mechanism of SDF-1/CXCR4 complex to attract autologous stem cells to areas of vascular insufficiency in order to help both increase collaterals and angiogenesis. VEGF also aids in new vascular endothelial formation with subsequent increase in blood vessels used as collaterals. The combined effect would be a continuous flow of stem cells into the area ultimately increasing the vascularity and blood flow to the area and therefore decreasing or even preventing cardiac ischemia in stented areas of the heart as well as repairing damaged myocardium.

The implant may be coated, impregnated, infused or otherwise coupled with such components. Alternatively, the stent may be coated with other types of drugs or therapeutic materials such as antibiotics, thrombolytics, anti-thrombotics, anti-inflammatories, cytotoxic agents, anti-proliferative agents, vasodilators, gene therapy agents, radioactive agents, immunosuppressants, chemotherapeutics, other endothelial cell attractors or promoters and/or stem cells. Such materials may be coated over all or a portion of the surface of the stent or the stent may have a porous structure or include apertures, holes, channels, or other features in which such materials may be deposited. Other embodiments of BES include xenografts, allografts or isografts comprising sleeve-like natural matrices derived from vessels of animal and human subjects including postmortem human donors as well as synthetically created matrices.

A protocol consisting of multiple doses of Gm-CSF or GCSF may be given either before or after stent placement in order to mobilize stem cells maximally from the bone marrow of the patient. The mobilization from bone marrow of endothelial progenitor cells (autologous stem cells from the patient), using either Gm-CSF or GCSF, substantially increases the number of stem cells homing on to such stents and areas downstream of the eluting stent. Optionally, stem cells may be injected into the patient after stem placement.

Maximizing the homing and the number of stem cells is the goal of this invention. Stem cells have a parakrine effect in that they release cytokines which are chemical factors that stimulate the live cells around them in the myocardial tissues of the heart. Studies have been done by others wherein stem cells are taken from the patient, concentrated, and then millions of stem cells are slowly injected or introduced into the coronary artery with a catheter while the blood flow is occluded so there is substantially no blood flow through the occluded vessel. Once the balloon catheter is removed, blood flow continues and the stem cells get diluted out because there is nothing to hold them in the area, i.e., there is not enough of a homing factor signal. After a short period of time, i.e., several days, there are no stem cells in the area. Such treatment only provides a slight improvement, i.e., about 3% in the left ventricular ejection fraction “LVEF”. In the worst patient populations, wherein they have such damage that their left ventricular ejection fraction is lower than 20% and their quality of life is poor, i.e., they cannot walk far, suffer from chronic ischemia, and have a life expectancy of three years or less; even a 3% increase improves their quality of life. However, this invention seeks enhanced LVEF over the known procedures.

This invention uses the treated implant, e.g., stent to assist in maintaining or holding the stem cells in the affected area for a much longer time while significantly increasing the number of available stem cells by the concomitant mobilization from the bone marrow immediately after the placement of the stent in the patient. Such procedure should significantly increase the number of stem cells mobilized into the peripheral circulation and via use of a continuous homing mechanism in the desired area via a stent or tissue or polymer eluting SDF-1/CXCR4 and VEGF, hold the stem cells in the needed location for a greatly extended period of time and subsequently improve the patient outcome, e.g., increased Left Ventricular ejection fraction “LVEF” via much improved angio/vasculogenesis, blood flow to ischemic areas as well as attracting or introducing large numbers of cardiac progenitor cells, and subsequently the quality of life and long term survival rate of this population of patients.

By the use of the phrase “ischemic tissue” is meant damaged tissue having a deficiency in blood or blood vessels typically as the result of myocardial ischemia and/or infarction, cerebrovascular ischemia, renal ischemia, pulmonary ischemia, limb ischemia, ischemic cardiomyopathy, ischemic organ (e.g., a transplanted organ) and myocardial ischemia. An individual in need of prevention, alleviation, and/or treatment of ischemia is prone to, suspected of having, or known to have tissue ischemic conditions such as those listed above. For example, individuals with circulatory problems due to diabetes or other conditions that damage circulation may be prone to or suspected of having ischemic tissue even if no such tissue has been observed directly. Tissues after organ transplant may also be prone to ischemia. Individuals with cardiovascular disease can be prone to both ischemia and myocardial infarction. The methods of this invention are suitable for tissue ischemia in a variety of animals including mammals.

### Coronary and Vascular Stents

The stent of the present invention is comprised of a stent body and a coating or bonding on the surface of the stent body or within the stent body of the aforementioned components. This invention contemplates the treatment and use of many types of stents known in the art as well as those that will be known. For example, such devices can include main artery stents, peripheral vascular stents, coronary artery stents, pulmonary artery stents, carotid artery stents, intracranial vascular stents, and aortic stent grafts, intracranial stents and renal stents which are all generally commercially available.

The composition for treating such stents can be used to coat all or part of the device, as may be beneficial for the vascular repair. For example, only the exterior of a stent could be coated to facilitate cell growth at the vascular wall. Furthermore, any device capable of use in contact with blood flow (particularly devices designed for long-term residence in contact with blood flow) can be used according to the invention. Specific, non-limiting examples of such devices include pacemaker leads, electrodes, myocardial patches, heart valves, and the like.

Generally, stents are rigid, or semi-rigid, tubular scaffoldings that are used to treat vessel narrowing or atherosclerosis. Specifically, atherosclerosis and other forms of coronary artery narrowing are treated with percutaneous transluminal angioplasty (“angioplasty”). The objective of angioplasty is to enlarge the lumen of an affected vessel by radial hydraulic expansion. The procedure is accomplished by inflating a balloon within the narrowed lumen of the affected artery. After (or during) such an angioplasty procedure, stents are deployed at the treatment site within the vessel to reduce the risks of reclosure. Stents are generally positioned across the treatment site, and then expanded to keep the passageway clear. The stent provides a scaffold which overcomes the natural tendency of the vessel walls of some patients to renarrow, thus maintaining the openness of the vessel and resulting blood flow.

Stents are typically either balloon expandable or self-expandable. Balloon expandable stents are mounted over a balloon or other expansion element on a delivery catheter. When the balloon is inflated, the balloon expands and correspondingly expands and deforms the stent to a desired diameter. The balloon can then be deflated and removed, leaving the stent in place. More specifically, a balloon-expandable stent comprises a metal tube, typically fabricated from stainless steel, chromium-cobalt alloy or other alloys, which is perforated in a pattern using a laser beam to add flexibility to the tube. To deliver a balloon-expandable stent, a surgeon places the stent over a balloon catheter, locates the catheter at the preselected target site in a damaged blood vessel, and expands the stent by applying pressure to the balloon catheter

A self-expanding stent is simply released from the delivery catheter so that it expands until it engages the vessel wall. Self-expanding stents are typically delivered to a treatment site while compressed or crimped within a constraining sheath. Retraction of the sheath removes the constraint and allows the stent to radially expand into engagement with the vessel wall. More specifically a self-expanding stent is a type of wire form typically made from Nitinol (nickel-titanium alloy) which has memory. This type of stent is placed over a catheter with a sleeve over the stent to hold it in a closed position. Once the target site is reached, the sleeve is removed and the stent springs open (self-expands).

This invention may also be used in association with drug eluting stents. Drug-eluting stents are stents coated with drugs that are slowly released.

This invention may also be used in association with biologically engineered stents. The term “biologically engineered stent” is meant to refer to a stent that incorporates a combination of sciences and technologies, e.g., biotechnology or medical science with biomedical engineering technology, all into one safe and efficacious medical device. A BES in accordance with the invention is a stent fabricated from a man-made material such as a metal and/or a polymer that further incorporates one or more biological components, i.e., components obtained from or derived from natural biological sources. Depending upon the application, a biological component of a BES, as the term is used herein, can encompass one or more of a wide range of components derived from living organisms, including, but not limited to: sleeves of biological materials derived from naturally occurring expandable biological conduits such as arteries, veins, and lymphatic vessels that are used, e.g. to cover one or more surfaces of a stent; stem cells that are incorporated into the stent before implantation; recombinant nucleic acids such as gene therapy vectors designed to locally deliver desired therapeutic genes to cells in the vicinity of a patient's stented blood vessel; proteins such as anti-bodies designed to attract endothelial progenitor cells (EPC) from the patient's circulation to encourage the establishment of an endothelial layer over the surface of the stent or various growth factors selected to promote the proliferation and differentiation of EPC into endothelial cells.

In one embodiment, the stent of this invention comprises a core and one or more coating layers. Materials suitable for fabricating the cores and coating materials of the stent are as described herein. Any one of the multi-drug and multi-section hybrid stents described can be configured in accordance with this invention by adding to the coating or polymer layers of the stent one or more biologics described herein that can promote the attraction, proliferation and differentiation of cells in the endothelial cell lineage, causing them to home to the area of the stent and to form a functional endothelial layer over the stent surface.

### Stem Cells

A “stem cell” is a cell from the embryo, fetus, or adult that has, under certain conditions, the ability to reproduce itself for long periods of time, or in the case of adult stem cells, throughout the life of the organism. It also can give rise to specialized cells that make up the tissues and organs of the body.

By the use of the term “stem cells” as used herein it is meant to include pluripotent stem cells, embryonic stem cells, multipotent adult stem cells, and progenitor and precursor cells.

A “pluripotent stem cell” has the ability to give rise to types of cells that develop from the three germ layers (mesoderm, endoderm, and ectoderm) from which all the cells of the body arise. Known natural sources of human pluripotent stem cells are those isolated and cultured from early human embryos from fetal tissue.

“Induced pluripotent stem cells” commonly abbreviated as iPS cells or iPSCs are a type of pluripotent stem cell artificially derived from a non-pluripotent cell—typically an adult somatic cell—by inducing a “forced” expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells have been made from adult stomach, liver, skin cells and blood cells. iPSCs were first produced in 2006 from mouse cells and in 2007 from human cells in a series of experiments by Shinya Yamanaka's team at Kyoto University, Japan, and by James Thomson's team at the University of Wisconsin-Madison. iPSCs are an important advance in stem cell research, as they may allow researchers to obtain pluripotent stem cells, which are important in research and potentially have therapeutic uses, without the controversial use of embryos.

An “embryonic stem cell” is derived from a group of cells called the inner cell mass, which is part of the early (4- to 5-day) embryo called the blastocyst. Once removed from the blastocyst the cells of the inner cell mass can be cultured into embryonic stem cells.

An “adult stem cell” is an undifferentiated (unspecialized) cell that occurs in a differentiated (specialized) tissue, renews itself, and becomes specialized to yield all of the specialized cell types of the tissue in which it is placed when transferred to the appropriate tissue. Adult stem cells are capable of making identical copies of themselves for the lifetime of the organism. This property is referred to as “self-renewal.” Adult stem cells usually divide to generate pro-genitor or precursor cells, which then differentiate or develop into “mature” cell types that have characteristic shapes and specialized functions, e.g., muscle cell contraction or nerve cell signaling. Sources of adult stem cells include but are not limited to bone marrow, blood, the cornea and the retina of the eye, brain, skeletal muscle, dental pulp, liver, skin, the lining of the gastrointestinal tract and pancreas.

The delivery or administration of stem cells to an individual includes the delivery or administration of exogenous stem cells as well as the mobilization of endogenous stem cells, as well as enhancing the bioavailability of spontaneously released endogenous stem cells.

Stem cells from the bone marrow are the most-studied type of adult stem cells. Currently, they are used clinically to restore various blood and immune components to the bone marrow via transplantation. There are currently identified two major types of stem cells found in bone marrow: hematopoietic stem cells (HSC, or endothelial progenitor cells) which are typically considered to form blood and immune cells, and stromal (mesenchymal) stem cells (MSC) that are typically considered to form bone, cartilage, muscle and fat. However, both types of marrow-derived stem cells have demonstrated extensive plasticity and multipotency in their ability to form the same tissues.

The marrow, located in the medullary cavity of bones, is the major site of hematopoiesis in adult humans. It produces about six billion cells per kilogram of body weight per day. Hematopoietically active (red) marrow regresses after birth until late adolescence after which time it is focused in the lower skull vertebrae, shoulder and pelvic girdles, tibiae, ribs, and sternum. Fat cells replace hematopoietic cells in the bones of the hands, feet, and arms (yellow marrow).

Means for isolating and culturing stem cells useful in the present invention are well known. Umbilical cord blood is an abundant source of hematopoietic stem cells. The stem cells obtained from umbilical cord blood and those obtained from bone marrow or peripheral blood appear to be very similar for transplantation use. Placenta is an excellent readily available source for mesenchymal stem cells. Moreover, mesenchymal stem cells have been shown to be derivable from adipose tissue and bone marrow stromal cells and speculated to be present in other tissues. Amniotic fluid and tissue is another excellent source of stem cells. While there are dramatic qualitative and quantitative differences in the organs from which adult stem cells can be derived, the initial differences between the cells may be relatively superficial and balanced by the similar range of plasticity they exhibit. For instance, adult stem cells both hematopoietic and mesenchymal, under the appropriate conditions can become myocardium cells.

“Totipotent stem cells” can grow and differentiate into any cell in the body, and thus can grow into an entire organism including placental tissues. These cells are not capable of self-renewal. In mammals, only the zygote and early embryonic cells are totipotent.

“Pluripotent stem cells” are true stem cells, with the potential to make any differentiated cell in the body, but cannot contribute to making the extra-embryonic membranes (which are derived from the trophoblast i.e. placenta).

“Multipotent stem cells” are clonal cells that self-renew as well as differentiate to regenerate adult tissues. “Multipotent stem cells” are also referred to as “unipotent” and can only become particular types of cells, such as blood cells or bone cells. Generally, the term “stem cells”, as used herein, refers to pluripotent stem cells capable of self-renewal.

### SDF-1/CXCR4

The coating or bonded compositions used herein on the stent are generally characterized as endothelialization factors such as autologous growth factor compositions. Normally, the endothelial cells that make up the portion of the vessel to be treated are quiescent at the time of stent graft implantation and do not multiply. As a result, the stent graft rests against a quiescent endothelial cell layer. If autologous growth factor compositions are administered to the treatment site with the stent graft deployment, the normally quiescent endothelial cells lining the vessel wall, and in intimate contact with the stent graft, will be stimulated to proliferate. The same will occur with smooth muscle cells and fibroblasts found within the vessel wall. As these cells proliferate they can grow into and around the stent graft lining such that the stent graft becomes physically attached to the vessel lumen rather than merely resting against it.

SDF-1 (or CXCL12) is a chemokine which is secreted by several tissues following exposure to hypoxia, in turn leading to the release of progenitor cells along a chemical gradient to the zone of tissue injury. Its receptor CXCR4, is a G-protein coupled receptor that is widely expressed on several tissues, including endothelial cells, smooth muscle cells, monocytes, hematopoietic and tissue committed stem cells. Binding of SDF-1 to CXCR4 induces several signal transduction pathways which regulate cell survival, stem cell homing and proliferation.

SDF-1, stromal derived factor 1/CXCR4 complex, is the stem/progenitor cell homing agent for the stem cells. SDF-1/CXCR4 produces the key cytokine that stimulates homing of CD34(+) EPCs into sites of ischemia. SDF-1 is a pro-regenerative agent or pro-angiogenic agent.

Such agents cause homing and proliferation and differentiation of endothelial progenitor cells. SDF-1 is a key stem cell mobilizer. While chemokines are particularly believed to be involved in signaling the recruitment of vascular progenitor cells, other signaling factors may also be involved in the process. Accordingly, in its broader aspects, any signaling factor that functions to stimulate or enhance recruitment of vascular progenitor cells at a site of vascular injury could be used according to the present invention.

Stromal cell-derived factor-1 (SDF-1), also known as pre-B cell growth-stimulating factor, is produced by bone marrow stromal cells and acts together with interleukin-7 as a co-mitogen for pre-B cells. SDF-1 has also been shown to be a chemokine which is chemotactic for different types of leukocytes. P-selectin, E-selectin, and L-selectin are cell adhesion molecules found in granules in endothelial cells and activated platelets and play a role in the recruitment of leukocytes to injury sites, particularly in vascular walls.

The stem cell homing factor bonded or incorporated within the stent, is preferably SDF-1/CXCR4 complex. The SDF-1/CXCR4 complex would be valuable to place in the infarcted region because the body's natural signaling to stem cells tapers off after a period of time, e.g., several days after a myocardial infarction due to the inflammation in the infarcted area. As the inflammation decreases, the body will stop signaling stem cells to home into the area. Therefore, by providing SDF-1/CXCR4 complex to the infarcted region within the stent that may degrade or elute over a pre-determined time period beyond the period of time that the signaling to stem cells to home into the region, may be extended beyond the initial period so that the regeneration of the myocytes in the infarcted region may occur.

When used in context of describing the invention, the terms “SDF-1” and “CXCR4” encompass all variants, alleles, analogs, isoforms, derivatives, species variants, fragments and the like.

CXCR4, also called fusin, is an alpha-chemokine receptor specific for SDF-1 and is a G-proteinlinked chemokine receptor. SDF-1 binds to CXCR4. The term “CXCR4”, as used herein, shall be understood to refer to the CXCR4 chemokine receptor, a receptor in the GPCR (G-protein coupled receptor) gene family, which is expressed by cells in the bone marrow, immune system and the central nervous system.

In response to binding its ligand SDF-1 (stromal cell-derived factor-1), CXCR4 is thought to trigger the migration and recruitment of immune cells, as well as the homing of stem cells (e.g., EPCs). The receptor is believed to enhance downstream signaling by several different pathways. As a GPCR, CXCR4 binding of SDF-1 activates G-protein mediated signaling, including downstream pathways such as ras, and PI3 kinase. PI3 kinase activated by SDF-1 and CXCR4 plays a role in lymphocyte chemotaxis in response to these signals. One endpoint of CXCR4 signaling is the activation of transcription factors such as AP-1 and chemokine regulated genes. JAK/STAT signaling pathways also appear to play a role in SDF-1/CXCR4 signaling.

### VEGF

Vascular endothelial growth factor (VEGF) is a signaling protein involved in both vasculoneogenesis and angiogenesis, i.e., a stem cell differentiation factor. It is part of the system that restores the oxygen supply to tissues when blood circulation is inadequate. Serum concentration of VEGF is high in Bronchial Asthma and low in Diabetes Mellitus. VEGF's normal function is to create new blood vessels during embryonic development, new blood vessels after injury, muscle following exercise, and new vessels (collateral circulation) to bypass blocked vessels. In vitro, VEGF has been shown to stimulate endothelial cell mitogenesis and cell migration. VEGF recruits endothelial cells and induces the formation of microcapillaries.

Other examples of proteins that may also be used as signaling factors for vascular progenitor cells according to the present invention include angiogenin, angiopoietin-1, del-1, fibroblast growth factors (e.g., acidic (aFGF) and basic (bFGF)), follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, and insulin-like growth factors (IGFs). All of the foregoing examples, and combinations thereof, may be used in the recruitment of vascular progenitor cells according to the invention.

The terms “VEGF -1” or “vascular endothelial growth factor-1” are used interchangeably to refer to a cytokine that mediates numerous functions of endothelial cells including proliferation, migration, invasion, differentiation of EPCs, survival, and permeability. VEGF is critical for angiogenesis.

In specific embodiments, the recombinant polypeptide comprises VEGF, BFGF, SDF, CXCR-4 or CXCR-5. Stem cells may be mobilized (i.e., recruited) into the circulating peripheral blood by means of cytokines, such as, for example, G-CSF, GM-CSF, VEGF, SCF (c-kit ligand) and bFGF, chemokines, such as SDF-1, or Interleukins, such as interleukins 1 and 8. Stem cells may also be recruited to the circulating peripheral blood of a mammal if the mammal sustains, or is caused to sustain, an injury. In one embodiment, the recombinant polypeptide is VEGF, BFGF, SDF, CXCR-4 or CXCR-5, or a fragment thereof which retains a therapeutic activity to the ischemic tissue.

As used herein the term “angiogenic protein” or related term such as “angiogenesis protein” means any protein, polypeptide, mutein or portion that is capable of, directly or indirectly, inducing blood vessel growth. Such proteins include, for example, acidic and basic fibroblast growth factors (aFGF and bFGF), vascular endothelial growth factor (VEGF-1), VEGF165, epidermal growth factor (EGF), transforming growth factor a and b (TGF-a and TFG-b), platelet-derived endothelial growth factor (PD-ECGF), platelet-derived growth factor (PDGF), tumor necrosis factor a (TNF-a), hepatocyte growth factor (HGF), insulin like growth factor (IGF), erythropoietin, colony stimulating factor (CSF), macrophage-CSF (M-CSF), granulocyte/macrophage CSF (GM-CSF), angiopoetin-1 (Ang1) and nitric oxide synthase (NOS). Preferred angiogenic proteins include vascular endothelial growth factors.

One of the first of these proteins was termed VEGF, now called VEGF-1. It exists in several different isoforms that are produced by alternative splicing from a single gene containing eight exons. Other vascular endothelial growth factors include VEGF-B and VEGF-C. Pro-angiogeneic agents for use in in this invention include, but are not limited to Vascular endothelial growth factor (VEGF)—A, B, C and D and receptors for the VEGF bone marrow stem cell agents that increase the production of stem cells such as Erythropoietin, Erythropoietin derivatives, BNP (Nesiritide), vascular endothelial growth factor (VEGF) agonists, Vascular endothelial growth factor (VEGF)—A, B, C and D, platelet-derived growth factor (PDGF)—AA, AB, BB, CC and DD, Fibroblast growth factor (FGF)—1, 2 and 4, Epidermal growth factor (EGF) as well as the receptors for the VEGF, PDGF, FGF and EGF receptors.

### Plerixafor

In a preferred embodiment, a composition which is administered to a patient, modulates expression and/or function for the SDF-1/CXCR4 axis. The compositions comprise nucleic acids, oligonucleotides, polynucleotides, peptides, polypeptides, enzymes, small molecules, organic or inorganic molecules and the like.

For example, and preferred, CXCR4 can be modulated by an antagonist such as AMD3100 (1,1′-[1,4-phenylenebis(methylene)]bis-1,4,8,11-tetraazacyclotetradecane), or mimetics thereof. AMD3100 (also known as PLERIXAFOR, rINN, USAN, MOZOBIL, JM 3100) is a symmetric bicyclam, prototype non-peptide antagonist of the CXCR4 chemokine receptor. Mimetics, such as for example, peptide or non-peptide antagonists can be designed to efficiently and selectively block the CXCR4 receptor.

Plerixafor can be used to augment mobilization of Endothelial Progenitor Cells (EPCs) or used for those patients that are poor mobilizors to G-CSF or GmCSF. The half-life of Prelixafor is about 8 hours; it thus should be administered 8 hours before stent placement.

During its activity it blocks the homing mechanism of the SDF-1/CXCR4 complex by binding to CXCR4 and blocking SDF-1, therefore it should also be given a few days, e.g., 3 days, after G-CSF or Gm-CS F administration.

Subsequent Protocol for Mobilization of Stem Cells

A protocol consisting of multiple doses of Gm-CSF or GCSF may be given intramuscularly (IM) before or after stent placement in order to mobilize stem cells maximally from the bone marrow of the patient. The mobilization from bone marrow of endothelial progenitor cells—autologous stem cells from the patient, using either Gm-CSF or GCSF, substantially increases the number of stem cells homing on to such stents. Agents which cause bone marrow stem cell efflux from the bone marrow are G-CSF, CXCR4 blocking agents (Plerixafor (rINN and USAN, also known as MOZOBIL, JM 3100 and AMD3100).

GM-CSF is a cytokine that functions as a white blood cell growth factor. GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection. The active form of the protein is found extracellularly as a homodimer.

Granulocyte colony-stimulating factor (G-CSF or GCSF) is a colony-stimulating factor hormone. GCSF is also known as colony-stimulating factor 3 (CSF 3). It is a glycoprotein, growth factor and cytokine produced by a number of different tissues to stimulate the bone marrow to produce granulocytes and stem cells. G-CSF then stimulates the bone marrow to release them into the blood.

Stem cells are obtained by concentrating them from the patient or by mobilizing them with an IM injection. In the preferred embodiment, the stem cells are mobilized from the bone marrow with an IM injection. They then subsequently home in on the stent that includes, inter alia, the SDF-1/CXCR4 homing factor. The antagonist effect of Plerixafor, which ultimately can block the homing mechanism, is avoided. The VEGF (vascular endothelial growth factor) on the stent also enhances re-vascularization of the area in order to allow more blood flow to the area, to decrease the ischemia.

Additionally and/or optionally, since stent emplacement is an invasive procedure for a patient, just prior to such procedure, obtain the patient's blood, obtain stem cells therefrom, then concentrate them and inject the stem cells after installation of the stent having the factors described herein embedded in the matrix. Thus, optionally you can have the stem cells immediately at the stent site after a prior injection of the Gm-CSF or GCSF. The treatment with only Gm-CSF or GCSF should have fewer side effects and would be just as effective as the combination treatment. It should be noted that GCSF is the stronger mobilizing agent.

Mobilizing stem cells of the bone marrow of the same patient, which peaks in about three days, used in combination with the stent of this invention, provides a continuous supply of mobilized stem cells held in the area by the homing factors used on or in the stent, i.e., for several weeks. Such a treatment provides enhanced left ventricular ejection fraction “LVEF” enabling the heart to function more efficiently because there is less of an ischemic area.

In particular, the invention methods are useful for therapeutic vasculogenesis for the treatment of myocardial ischemia in humans. For example, the methods described herein for treatment of myocardial ischemia can be used in conjunction with coronary artery bypass grafting or percutaneous coronary interventions. The methods described herein are particularly useful for subjects that have incomplete revascularization of the ischemic area after surgical treatments and, therefore, have areas of ischemic but viable myocardium. Subjects that can significantly benefit from the therapeutic vasculogenesis according to the methods of the invention are those who have large areas of viable myocardium jeopardized by the impaired perfusion supplied by vessels that are poor targets for revascularization techniques. Other subjects that can benefit from the therapeutic vasculogenesis methods are those having vessels of small caliber, severe diffuse atherosclerotic disease, and prior revascularization, in particular bypass grafting. Therefore, the therapeutic vasculogenesis according to the methods of the invention can particularly benefit subjects with chronic myocardial ischemia.

Individuals who benefit from the method and stent described herein are people who are suffering from or experiencing a cardiovascular event, such as intractable myocardial ischemia or acute myocardial infarction (AMI). Acute myocardial infarction is commonly known as a “heart attack.”

The term “myocardial infarction” relates to changes in the heart muscle (myocardium) that occur due to the sudden deprivation of circulating blood, caused by events such as arteriosclerosis (narrowing or clogging of the coronary arteries) and thrombosis (clot), which reduce the flow of oxygenated blood to the heart. The main change is death (necrosis) of myocardial tissue, which can lead to permanent damage or death of the heart muscle.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the compositions, chemicals, instruments, statistical analyses and methodologies which are reported in the publications which might be used in connection with the invention for proof of concept. All references cited in this specification are to be taken as indicative of the level of skill in the art. Nothing herein is to be construed as an admission that the invention is not entitled to antedate such disclosure by virtue of prior invention.

While the invention has been described in connection with what is presently considered to be practical and preferred embodiments thereof, it should be understood that it is not to be limited or restricted to the disclosed embodiments, but rather is intended to cover various modifications, substitutions and combinations within the spirit and scope of the appended claims. In this respect, one should also note that the protection conferred by the claims is determined after their issuance in view of later technical developments and would extend to all legal equivalents.

What is claimed is:

1. A biologically engineered stent for implanting in a vessel upstream to and proximal a damaged myocardium of a patient, the biologically engineered stent having bonded thereto or incorporated therein a vascular endothelial growth factor (VEGF).

2. The biologically engineered stent of claim 1, wherein the VEGF is VEGF-1.

3. A biologically engineered stent for implanting in a vessel upstream to and proximal a damaged myocardium of a patient, the biologically engineered stent having bonded thereto or incorporated therein a signaling factor for vascular progenitor cells selected from the group consisting of angiogenin, angiopoietin-1, del-1, fibroblast growth factors, follistatin, granulocyte colony-stimulating factor (G-CSF), hepatocyte growth factor (HGF), scatter factor (SF), Interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), vascular permeability factor (VPF), Complement Components, or insulin-like growth factors (IGFs).

4. A biologically engineered stent for implanting in a vessel upstream to and proximal a damaged myocardium of a patient, the biologically engineered stent having bonded thereto or incorporated within the biologically engineered stent a stem cell homing factor.

5. The biologically engineered stent of claim 4, wherein the stem cell homing factor is SDF-1/CXCR4 complex.

6. A biologically engineered stent for implanting in a vessel upstream to and proximal a damaged myocardium of a patient to stimulate survival and repair of the damaged myocardium, the biologically engineered stent comprising an amount of a component selected from the group consisting of:
a. A component that functions as a homing mechanism for stem cells to the damaged myocardium, or
b. A component that functions as a signaling factor to signal the recruitment of vascular progenitor cells to the damaged myocardium; or
c. Mixtures of i. and ii.

7. A biologically engineered stent for implanting in a vessel upstream to and proximal a damaged myocardium of a patient to stimulate survival and repair of the damaged myocardium, the biologically engineered stent comprising an amount of a component selected from the group consisting of :
a. Stromal Derived Factor (SDF1)/CXCR4 complex, or
b. Vascular Endothelial Growth Factor (VEGF), or
c. Mixtures of a. and b.

8. A biologically engineered stent for implanting in a vessel upstream to and proximal a damaged myocardium of a patient to stimulate survival and repair of the damaged myocardium, the biologically engineered stent impregnated with Stromal Derived Factor (SDF1)/CXCR4 complex and Vascular Endothelial Growth Factor (VEGF), to promote autologous stem cell growth around the biologically engineered stent and revascularization around and downstream of the biologically engineered stent.

9. The biologically engineered stent of claim 7, wherein the biologically engineered stent is further coated, impregnated, infused or otherwise coupled with the gene therapy vector.

10. The biologically engineered stent of claim 7, wherein the biologically engineered stent is a xenograft, an allograft or an isograft.

11. The biologically engineered stent of claim 7, wherein the biologically engineered stent further includes a component selected from the group consisting of an antibiotic, a thrombolytic, an anti-thrombotic, an anti-inflammatory, a cytotoxic agent, an anti-proliferative agent, a vasodilator, a gene therapy agent, a radioactive agent, an immunosuppressant, a chemotherapeutic, an endothelial cell attractor or promoter, stem or mixtures thereof.

12. The biologically engineered stent of claim 7, wherein the biologically engineered stent is a main artery biologically engineered stent, a peripheral vascular biologically engineered stent, a coronary artery biologically engineered stent, a carotid artery biologically engineered stent, a pulmonary artery biologically engineered stent, an intracranial vascular biologically engineered stent, an aortic biologically engineered stent graft, an intracranial biologically engineered stent or a renal biologically engineered stent.

13. A method of treating a damaged myocardium in a cardiac circulatory system to stimulate survival and repair of the myocardium in a patient having such damaged myocardium, comprising:
a. Providing a biologically engineered stent of claim 7 for the treatment of the damaged myocardium,
b. Inserting the biologically engineered stent in a vessel upstream to and proximal the damaged myocardium of a patient;
Whereby the biologically engineered stent promotes the local production of therapeutic factors that attract stem cells and enhance the formation of collateral vessels as well as attracting cardiac progenitor cells to a damaged myocardium, thereby stimulating survival and repair of the damaged myocardium.

14. A method of treating damaged myocardium to stimulate survival and repair of the myocardium in a patient having such damaged myocardium comprising:
a. Providing the biologically engineered stent of claim 8 for the treatment of the damaged myocardium;
b. Inserting the biologically engineered stent in a vessel upstream to and proximal the damaged myocardium of the patient;
Whereby the biologically engineered stent promotes the local production of therapeutic factors that attract stem cells and enhance the formation of collateral vessels as well as attracting cardiac progenitor cells to a damaged myocardium, thereby stimulating survival and repair of the damaged myocardium.

15. The method of claim 14, wherein subsequent to inserting the biologically engineered stent into the vessel, treating the patient with at least one dose Gm-CSF or GCSF to mobilize stem cells maximally from the bone marrow of the patient.

16. The method of claim 14, wherein subsequent to or prior to inserting the biologically engineered stent into the vessel, treating the patient intramuscularly (IM) with at least one dose Gm-CSF or GCSF to mobilize stem cells maximally from the bone marrow of the patient.

17. The method of claim 14, further comprising subsequent to treating the patent with at least one dose Gm-CSF or GCSF, injecting stem cells into the patient.

18. The method of claim 14, wherein prior to inserting the biologically engineered stent into the vessel treating the patient with AMD3100 (1,1′-[1,4-phenylenebis(methylene)]bis-1,4,8,11-tetraazacyclotetradecane) or mimetics thereof.

19. The method of claim 17, wherein the stem cells are induced pluripotent stem cells.

20. An implant for implanting in a patient, the implant having bonded thereto Stromal Derived Factor (SDF1)/CXCR4 complex and Vascular Endothelial Growth Factor (VEGF) to continuously attract autologous stem cell to the implant or downstream of the implant.

21. The implant of claim 20, wherein the implant is a pacemaker lead, an electrode, a myocardial patch, or a heart valve.
